(19) United States
(12) Patent Application Publication
(10) Pub. No.: US 2006/0069334 A1
(43) Pub. Date: Mar. 30, 2006

(54) Compact universal splint apparatus for focused immobilization such as of a single digit of the foot or hand, and method

(76) Inventor: Barry M. Moskowitz, Aventura, FL (US)

Correspondence Address: FRANK L. KUBLER, 13261 S.W. 54th Court, Miramar, FL, 33027, US

(21) Appl. No.: 10/949,656

(22) Filed: Sep. 24, 2004

(51) Int. Cl. A61F 5/00

(52) U.S. Cl. 602/5; 602/22; 602/30

(57) ABSTRACT

A splint apparatus includes an immobilization member which is substantially rigid; and an apparatus mounting structure including a flexible sheet and a immobilization member retaining structure removably retaining the immobilization member and body area engaging means. The immobilization member preferably is an immobilization panel. The body area engaging structure preferably includes at least one arm portion extending from the flexible sheet and having an arm portion free end for wrapping around a part of a human body, and having a arm portion fastening structure for removably securing the at least one arm portion remote end after the at least one arm portion is wrapped around a part of a human body. A method of using the splint apparatus to splint a part of a human body is also provided.

### BACKGROUND OF THE INVENTION

1. Field of the Invention

The present invention relates generally to the field of medical appliances for placement on the human body. More specifically the present invention relates to an open universal splint apparatus having an apparatus mounting structure and a rigid immobilization member such as a thin immobilization panel sized in length and width and otherwise shaped to properly fit and individually immobilize virtually any foot or hand digit or adjoining body structure without immobilizing an adjacent digit, with easy splint apparatus application and removal.

The apparatus mounting structure preferably is generally cross-shaped and for many applications is T-shaped, including a member retaining portion and a body area engaging portion in the form of two arm portions protruding from the member retaining portion for wrapping around a part of the human body, and preferably formed of elastic sheet material. The immobilization panel is secured relative to the member retaining portion of the T-shape, and the member retaining portion is placed along the length of digit, which may be either a finger or a toe or adjoining bony structure, and the body area engaging portion is wrapped around the distal end of the digit and secured with fastening means such as hook and loop fastener strips. The immobilization panel preferably is removably inserted into a pocket formed in the member retaining portion. Where an adjustment in size is needed, the present splint construction permits removal of the immobilization panel so that it can be trimmed to a suitable length and width for the individual patient and the particular digit or other body area, and then inserted into the member retaining portion pocket. The member retaining portion does not itself wrap around the digit because the digit may swell as a result of the injury. The apparatus is intended for splinting the phalanges of both the feet and the hands, but also is intended to fit around the foot to splint the metatarsals, which are immediately behind the toes, and to fit around the palm of the hand to splint the metacarpals, which are immediately behind the fingers.

A method of using the splint apparatus where size adaptation is required includes the steps of removing the panel from the pocket; trimming the panel with cutting means such as scissors to a suitable size and shape; inserting the panel into the pocket; placing the member retaining portion against and along the body part to be splinted, and wrapping the body area engaging portion around the body part and releasibly fastened with the fastening means.

2. Description of the Prior Art

There have long been splints for applying to various injuries, and most commonly to fractures. Problems with these prior splints have been that they are not useful for more than one body area such as for both toes and fingers, they can be time consuming to apply, they are uncomfortable to the patient and difficult to adapt to the structure to be splinted, and they sometimes obstruct swelling and circulation. Another problem has been that the size and shape of the splint is has been difficult to adapt to fit the particular digit or other body area of the individual patient, partly because of the materials used to construct prior splints. Still another problem has been that some prior splints have been expensive to manufacture.

Noble, U.S. Pat. No. 6,575,925, issued on Jun. 10, 2003, reveals a finger splint. Noble includes a substantially inflexible supporting shell formed of aluminum, plastic or foam and including a protruding retaining member loop, and at least one securing strap for fitting through the loop and wrapping around the finger to retain the supporting shell.

Bracamonte-Sommer, U.S. Pat. No. 6,478,761, issued on Nov. 12, 2002, discloses a rollable body part protector. The protector includes a sheet constructed to be sufficiently stiff in one direction to resist bending of the body part and yet sufficiently flexible in another direction so that it can be rolled easily around the body part, and includes fastening means for securing the splint around the body part. The sheet may be a fabric having fibers integrated with the weave to impart stiffness in one direction.

Ogle, II, U.S. Pat. No. 4,644,941, issued on Feb. 24, 1987, teaches an orthopedic splint for immobilization of an injured body member such as a finger or toe. The splint includes a ring-like base portion sized to fit around a digit close to the digit proximal end and several generally rigid elongate support members extending distally from the base portion along the body member and spaced laterally from each other. A flexible strap wraps around the elongate support members to adjust the diameter of the splint.

Lazarian, U.S. Pat. No. 4,899,737, issued on Feb. 13, 1990, reveals a splint for complete circumferential immobilization of an extremity or a terminal member of an extremity. Lazarian includes a substantially cylindrical elastic outer member, several stiffening members inside the elastic outer member and a liner located inside the elastic outer member and the stiffening members to prevent bending of the extremity or of the terminal member of the extremity.

Lockhart, U.S. Pat. No. 5,957,875, issued on Sep. 28, 1999, reveals a toe splint for an outer toe, including a molded flat plastic shell having a supporting part which may be curved in cross-section to fit partly around an injured digit and an integral and contiguous holding part sized proximally to extend over part of the foot behind the toe, and including securing means in the form of adhesive tape for wrapping around the shell and the foot.

Grasinger, U.S. Pat. No. 5,230,699, issued on Jul. 27, 1993, teaches a splint for a fractured phalanx. Grasinger includes a hinged splint body having a hinging portion for positioning beneath a joint immediately adjacent and distal to the fractured phalanx and a fulcrum positioned between the splint body and the finger directly beneath the fractured phalanx line of fracture, and includes means for securing the splint body and fulcrum in place. The splint body is free to move about the hinged portion to follow the portion of the finger distal to the fractured phalanx, so that an upward reduction force is exerted on the fractured phalanx at the fracture line by the fulcrum.

Ewing, U.S. Pat. No. 3,692,022, issued on Sep. 19, 1972, discloses a digital splint for use on a finger. Ewing includes a rigid tubular member for placement around a finger and having a series of opposing slots along its length for receiving opposing portions of a spring clip which engage the finger. A problem with Ewing is that the splint does not permit the finger to enlarge in diameter with swelling.

Fuzak, U.S. Pat. No. 3,529,597, issued on Sep. 22, 1970, reveals a fingertip bandage. Fuzak includes a flexible panel radially cut into opposing wing segments, so that the panel can be wrapped over the tip of a finger and the wing portions pivoted around and against the sides of the finger wherein they are secured with an adhesive.

Hurney, U.S. Pat. No. 3,476,109, issued on Nov. 4, 1969, teaches a fingertip bandage. Hurney includes a bandage dispensing tube containing a flexible cup-shaped bandage, so that a fingertip inserted into the tube bears against and turns the cup-shaped bandage inside out engagingly around the fingertip. A Brown, U.S. Pat. No. 3,330,270, issued on Jul. 11, 1967, discloses a finger splint for holding a finger in a set linear position and permitting movement of the injured finger from the metacarpal-phalangeal joint. Brown includes a support structure in the form of elongate wire loop secured against a pad of resilient sheet material and taped to the injured finger. The portion of the sheet material extending across a wider circular portion of the wire loop fits over deflects to receive the knuckle at the base of the injured finger.

Chandler, U.S. Pat. No. 3,039,460, issued on Jun. 19, 1962, teaches a finger splint. Chandler includes a splint body having a peripheral shape with convex side edges and formed of two layers of flexible fabric adhesively jointed together face to face, one layer having a central semi-elliptical cut forming a flap, and flexible stiffening elements along the edges of the body and adhesive straps extending outwardly from the side edges for wrapping around a finger.

It is thus an object of the present invention to provide a splint apparatus which has substantially universal application in that it has an open configuration and is pre-sized and shaped to fit virtually any digit of the foot or hand or adjacent body structure and which is not tied to and does not in any other way immobilize an adjacent digit or other body structure.

It is another object of the present invention to provide such a splint apparatus which is quickly and easily applied and removed by an elderly or handicapped patient or office worker with no experience and virtually no explanation, which can be removed and sterilized, and which is sufficiently inexpensive for removal and replacement with each office visit.

It is still another object of the present invention to provide such a splint apparatus which is sufficiently compact that when applied to a digit or other structure of the foot, both the foot and splint fit into a shoe.

It is still another object of the present invention to provide such a splint apparatus which automatically adapts in size to snugly fit digits of various diametric sizes and to fit the same digit when the digit has changed in diametric size due to swelling with adjustably overlapping elastic arm portions.

It is yet another object of the present invention to provide such a splint apparatus which is adaptable length and width to fit a particular digit or other body area of an individual patient.

It is an additional object of the present invention to provide such a splint apparatus which is comfortable to the patient and open to further permit expansion due to swelling caused by an injury and thus permitting circulation.

It is finally an object of the present invention to provide such a splint apparatus which is inexpensive to manufacture.

### SUMMARY OF THE INVENTION

The present invention accomplishes the above-stated objectives, as well as others, as may be determined by a fair reading and interpretation of the entire specification.

A splint apparatus is provided including an immobilization member which is substantially rigid; and a mounting structure including a flexible sheet and immobilization member retaining portion removably retaining the immobilization member and body area engaging portion. The immobilization member preferably is an immobilization panel.

A splint apparatus is further provided, including an immobilization panel which is substantially rigid and thin. In circumstances in which splint apparatus size adjustment is needed, the immobilization panel can be cut with a cutting device to dimensions suited for a particular application; and a mounting structure including a flexible sheet and an immobilization panel retaining portion removably retaining the immobilization panel, and including a body area engaging portion. The immobilization panel retaining portion preferably includes a pocket in the sheet material sized to receive at least part of the immobilization panel. The body area engaging portion preferably includes a portion of the flexible sheet in the form of at least one arm portion having an arm portion free end for wrapping around a part of a human body, and having an arm portion fastening mechanism for removably securing the at least one arm portion remote end after the at least one arm portion is wrapped around a part of a human body.

In the event that the splint apparatus requires size alteration, a method is provided of splinting a part of a human body using a splint apparatus including an immobilization member which is substantially rigid; and a mounting structure including a flexible sheet and an immobilization member retaining structure removably retaining the immobilization member and a body area engaging portion including at least one arm portion having an arm portion free end and an arm portion fastening mechanism, including the steps of: removing the immobilization member from the pocket; trimming the immobilization member with a trimming device to desired immobilization member dimensions; inserting the immobilization member into the pocket; placing the member retaining portion against a human body part to be splinted; wrapping the at least one arm portion around the part of the human body; and releasibly fastening the at least one arm portion in position around the part of the human body with the fastening mechanism.

### BRIEF DESCRIPTION OF THE DRAWINGS

Various other objects, advantages, and features of the invention will become apparent to those skilled in the art from the following discussion taken in conjunction with the following drawings, in which:

FIG. 1 is a plan view of a preferred embodiment of the splint apparatus.

FIG. 2 is a perspective view of the splint apparatus being fitted onto a toe.

FIG. 3 is a side view of a foot with the splint apparatus immobilizing a toe as in FIG. 2.

FIG. 4 is a bottom view of a foot showing the splint apparatus arm portions engagingly fitted around a toe to immobilize the toe.

FIG. 5 is a top view of the foot of FIG. 4.

FIG. 6 is a bottom view of a foot showing the splint apparatus arm portions engagingly fitted around the body of a foot to immobilize a metatarsal, which is the structure immediately behind a toe.

FIG. 7 is a top view of the foot of FIG. 6.

FIG. 8 is a bottom view of a hand showing the palm and fingers with the splint apparatus arm portions engagingly wrapped around one of the fingers to immobilize the individual finger.

FIG. 9 is a top view of the top of the hand of FIG. 8.

FIG. 10 bottom view of a hand showing the palm and fingers with the splint apparatus arm portions engagingly wrapped around the palm to immobilize an individual metacarpal.

FIG. 11 is a top view of the hand of FIG. 10.

### DETAILED DESCRIPTION OF THE PREFERRED EMBODIMENTS

As required, detailed embodiments of the present invention are disclosed herein; however, it is to be understood that the disclosed embodiments are merely exemplary of the invention which may be embodied in various forms. Therefore, specific structural and functional details disclosed herein are not to be interpreted as limiting, but merely as a basis for the claims and as a representative basis for teaching one skilled in the art to variously employ the present invention in virtually any appropriately detailed structure.

Reference is now made to the drawings, wherein like characteristics and features of the present invention shown in the various FIGURES are designated by the same reference numerals.

### First Preferred Embodiment

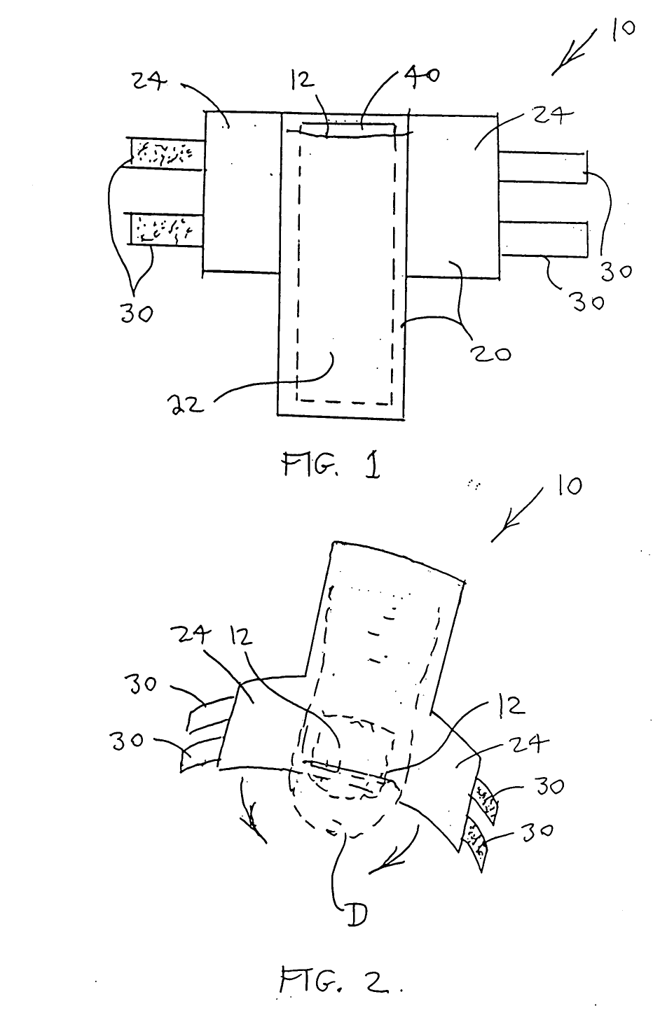

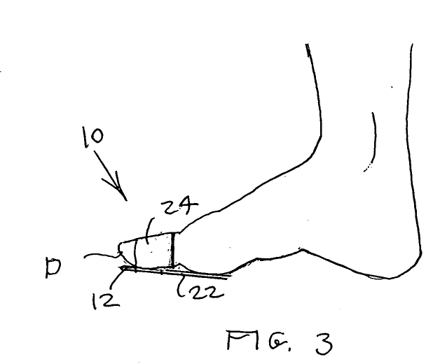

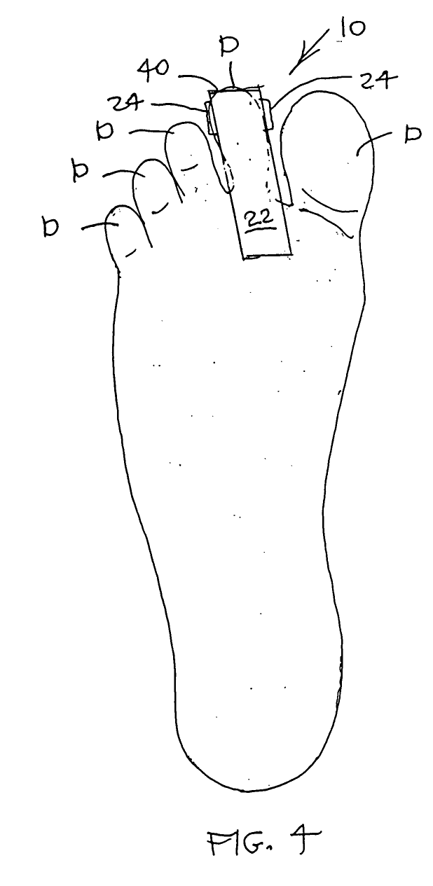

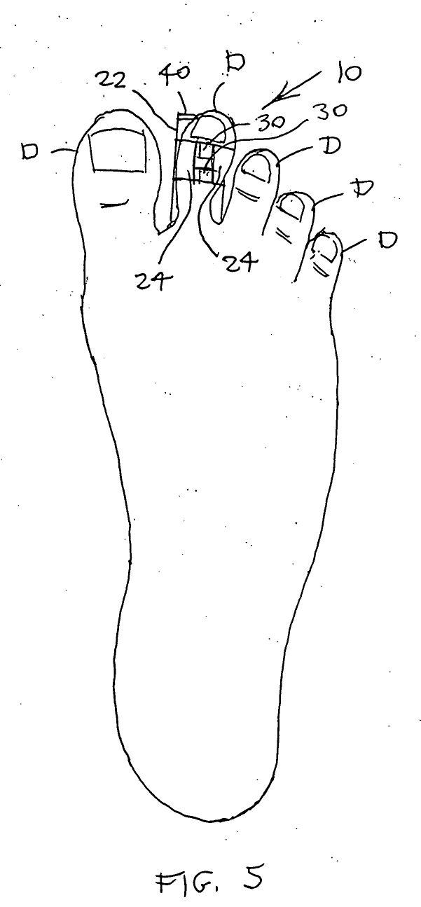

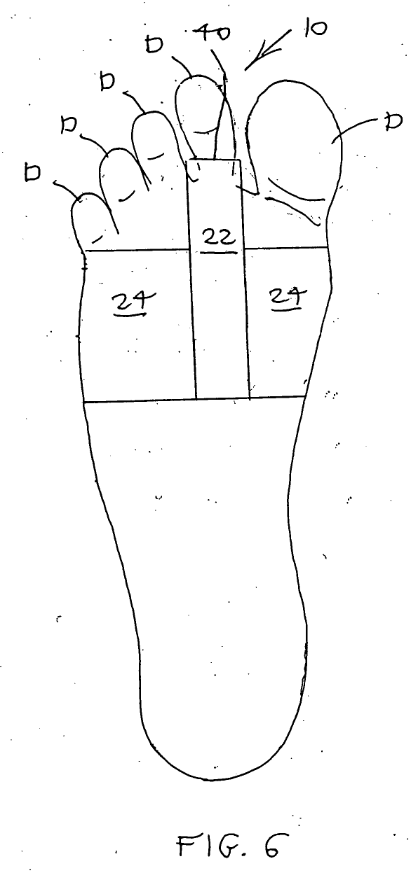

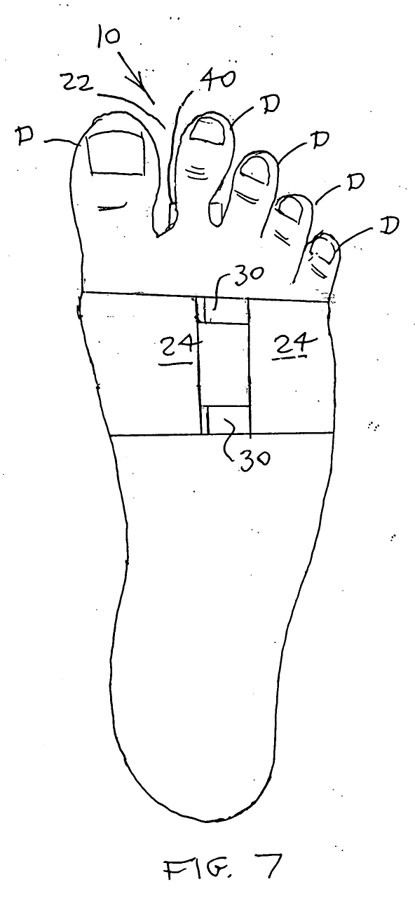

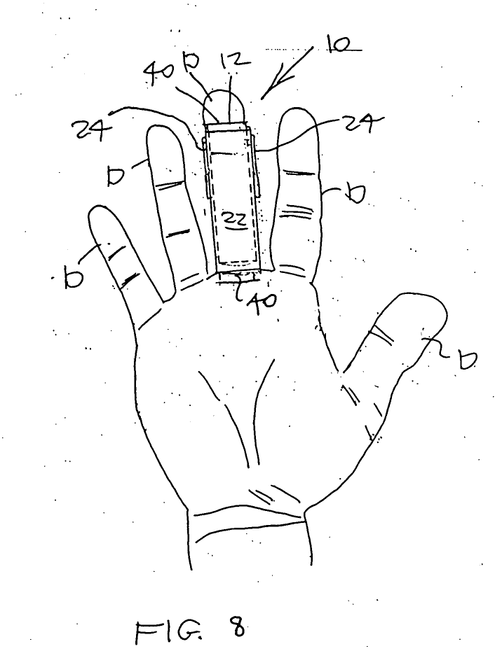

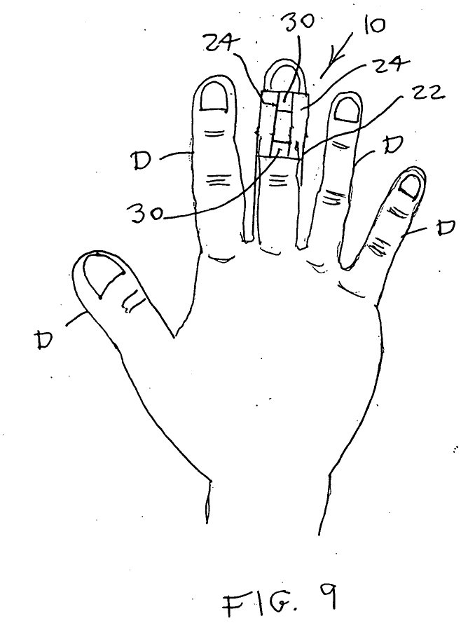

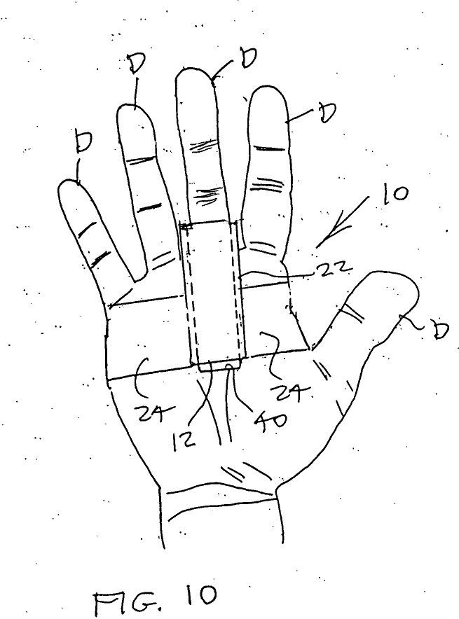

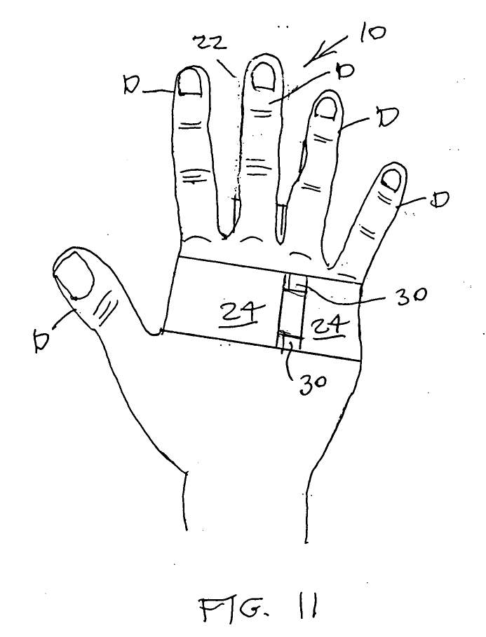

Referring to FIGS. 1-11, an open splint apparatus 10 is disclosed, including an apparatus mounting structure 20 and a substantially rigid (for purposes of the present specification and claims meaning either rigid or semi-rigid) immobilization member in the form of a thin immobilization panel 40 which can be removed from the apparatus mounting structure 20 for meeting virtually any immobilization need, such as for fracture. Apparatus mounting structure 20 preferably is substantially cross-shaped and for many applications is T-shaped, having a member retaining portion 22 and a body area engaging portion in the form of two arm portions 24 protruding from the member retaining portion 22 for wrapping around part of the human body, and preferably is formed of elastic sheet material to permit expansion and contraction with swelling size changes. Immobilization panel 40 is secured along the member retaining portion 22, and the member retaining portion 22 is placed along the length of digit D, which may be a toe, a finger or other body structure. The arm portions 24 are wrapped around the distal end of the digit D and secured with fastening means 30 such as overlapping face to face hook and loop fastener strips secured to arm portion free ends 24a. This construction permits adjustment of the extent of fastener strip 30 overlap to thereby adjust the resultant arm portion 24 loop diameter and thus the elastic tightness of the arm portions 24 around a digit. This size adjustment is automatic each time splint apparatus 10 is applied to a digit, since the diameter of the digit automatically determines the size of the arm portion 24 loop as the arm portions 24 are wrapped around the digit. Immobilization panel 40 is removably inserted into a pocket 12 formed in the member retaining portion 22. This construction permits removal of the immobilization panel 40 in the event that panel 40 needs to be trimmed to a suitable length and/or width differing from the pre-cut substantially universal size in order to fit an individual patient and a particular digit D or other body area, panel 40 is removed from member retaining portion 12 and cut with an instrument such as scissors or a knife and then reinserted into the member retaining portion 12. The member retaining portion 22 does not wrap around the digit D because full enclosure could obstruct expansion of the digit with swelling. Apparatus 10 is primarily intended for splinting the phalanges of both the feet and the hands. Yet apparatus 10 is also intended to fit around the foot to splint the metatarsals, which are immediately behind the toes as shown in FIGS. 6 and 7, and to fit around the palm of the hand to splint the metacarpals as shown in FIGS. 10 and 11, which are immediately behind the fingers.

### Method

In practicing the invention, the following method may be used. In the event that splint apparatus 10 requires size alteration, the panel 40 is removed from the pocket 12; the panel 40 is trimmed with cutting means such as scissors to a desired size and shape; the panel 40 is inserted into the pocket 12; the member retaining portion 22 is placed against and along the body part to be splinted, and the arm portions 24 are wrapped around the body part and releasibly fastened with the fastening means 30. Arm portions 24 can be positioned either forwardly or rearwardly along the limb or digit D, depending on where the injury is located, so that the member retaining portion 22 may or may not reach a digit D.

While the invention has been described, disclosed, illustrated and shown in various terms or certain embodiments or modifications which it has assumed in practice, the scope of the invention is not intended to be, nor should it be deemed to be, limited thereby and such other modifications or embodiments as may be suggested by the teachings herein are particularly reserved especially as they fall within the breadth and scope of the claims here appended.

1. A splint apparatus, comprising:
an immobilization panel which is pre-formed, substantially rigid, thin and of substantially universal size for splinting human digits;
and an apparatus mounting structure comprising a flexible and elastic sheet and immobilization member retaining means removably retaining said immobilization member and body area engaging means;
wherein said body area engaging means comprises at least one arm portion extending from said flexible sheet and having an arm portion free end for wrapping around a part of a human body, and having arm portion fastening means for removably securing the at least one arm portion remote end after said at least one arm portion is wrapped around a part of a human body.

2. (canceled)

3. (canceled)

4. A splint apparatus, comprising:
an immobilization panel which is pre-formed, substantially rigid and thin, and can be cut with cutting means to dimensions suited for a particular application;
and an apparatus mounting structure comprising flexible sheet and an immobilization panel retaining portion comprising a pocket in said sheet material sized to receive at least part of said immobilization panel, and comprising a body area engaging portion;
wherein said body area engaging portion comprises a portion of said flexible sheet in the form of at least one arm portion having an arm portion free end for wrapping around a part of a human body, and having arm portion fastening means for removably securing the at least one arm portion remote end after said at least one arm portion is wrapped around a part of a human body.

5. (canceled)

6. (canceled)

7. A method of splinting a digit of a human body, comprising the steps of:
providing a splint apparatus comprising a pre-formed thin immobilization panel which is substantially rigid and of substantially universal size for splinting human digits, and an apparatus mounting structure comprising a flexible sheet and immobilization panel retaining portion comprising a pocket in the flexible sheet sized to receive at least part of the immobilization panel and body area engaging means comprising at least one arm portion having an arm portion free end and arm portion fastening means;
placing at least part of the immobilization panel into the pocket to extend along and against the digit to be splinted;
and releasibly fastening the at least one arm portion around a digit of the human body with the fastening means.

8. The method of claim 7, comprising the additional steps of:
removing the immobilization panel from the pocket;
trimming the immobilization panel with trimming means to desired immobilization panel dimensions;
and inserting the immobilization panel into the pocket.
